(19) United States
(12) Patent Application Publication
(10) Pub. No.: US 2025/0325595 A1
(43) Pub. Date: Oct. 23, 2025

(54) MIXTURES OF PLACENTAL PARTICLES, GRAFTS INCLUDING THEM, AND METHODS FOR MAKING AND USING THEM

(71) Applicant: Musculoskeletal Transplant Foundation, Edison, NJ (US)

(72) Inventors: Kevin WU, Morganville, NJ (US); Marzena Kononiuk, Edison, NJ (US); Scott Lansdale, Bordentown, NJ (US); Alison Ling, Piscataway, NJ (US); Yucheng Guan, Edison, NJ (US); Carlos A. Zabala, Edison, NJ (US); German Eduardo Jurado Rivera, Rahway, NJ (US)

(21) Appl. No.: 19/183,186

(22) Filed: Apr. 18, 2025

Related U.S. Application Data
(60) Provisional application No. 63/635,831, filed on Apr. 18, 2024.

(51) Int. Cl. A61K 35/50; C12N 5/073

(52) U.S. Cl. CPC A61K 35/50; A61K 35/51; A61L 27/3604; A61L 27/54; C12N 5/0605; C12N 2509/10

(57) ABSTRACT

Placental particle (PP) compositions are provided which comprise a mixture of placental particles derived from each of amnion membrane and chorionic membrane, and optionally also umbilical cord. For PP compositions comprising particles of all three of the aforesaid placental tissue types, the PP composition has the following particle size distribution: about 50%-80% of particles from about 5 to about 80 um, about 10%-40% of particles from greater than about 80 to about 150 um, and about 15%-25% of particles greater than about 150 um, based on the total population of placental particles present in the PP composition. The aforesaid PP compositions are applied as a wound covering to provide better (i.e., more thorough and complete) wound coverage than placental particle mixtures without the aforesaid particle size distribution. Methods of making the PP compositions are also provided.

### CROSS-REFERENCE TO RELATED APPLICATIONS

The present application claims the benefit of and priority, under 35 U.S.C. § 119(e), to U.S. Provisional Patent Application No. 63/635,831, filed Apr. 18, 2025, the entire disclosure of which is incorporated by reference herein.

### FIELD OF THE INVENTION

The invention described and contemplated herein relates to placental particle compositions which comprise a mixture of dehydrated placental particles derived from one or more of amnion, chorion, umbilical cord, and chorionic plate, methods of making such placental particles and mixtures thereof, as well as and methods of making and using such compositions as grafts, with or without additional materials.

### BACKGROUND

Different types of placental tissues, as well as combinations of them, have been successfully and beneficially used to treat various conditions for several decades. For example, the low immunogenicity and enhanced healing, anti-inflammatory, and anti-adhesion properties of amniotic membrane have been known for at least 50 years. More recently, it has been recognized that chorion membrane also has enhanced healing and anti-inflammatory properties. Umbilical cord shares at least some of the same properties as amniotic and chorionic membranes, as well as providing a bulking or volumizing capability.

The biological mechanisms and pathways for operation of the aforesaid properties are still being studied and understood. Nonetheless, placental tissues have been used for treatment of wounds, burns, and ulcers of the skin and eyes, as well as treatment of inflammation and osteoarthritis of the joints and post-operative healing and scar reduction. Most recently, the use of human placental tissue is being explored for treatment of chronic and difficult-to-heal wounds and soft tissue injuries. Umbilical cord is currently studied for use in the field of regenerative medicine to treat injuries and chronic, degenerative conditions.

Many physical forms, derivatives, and combinations of one or more placental tissues have also been developed, studied and successfully used in the aforementioned treatments. Some physical forms, derivatives, and combinations are more beneficial or effective than others, depending on the particular condition and body region being treated. Accordingly, research continues for studying and developing improved and optimized placental compositions in the form of sheets, patches, particulates of various shapes and sizes, as well as suspensions and extracts.

In particular, the use of one or more types of placental tissues in a particulate physical form has provided benefits to diseased and injured tissue. However, the processing, formulation and clinical usage of placental particles continues to be improved and optimized to better meet the needs of patients in alleviating pain and treating other symptoms of musculoskeletal disorders and other medical conditions. The invention described and contemplated herein provides improved placental tissue compositions comprising mixtures of placenta derived particles for use in the medical and surgical fields.

### SUMMARY

A composition is provided comprising placenta-derived particles (PP composition) which comprises a mixture of one or more of: amnion membrane derived particles (APs), chorionic membrane derived particles (CPs), umbilical cord derived particles (UCPs), and chorionic plate derived particles (CPPs). The PP composition includes from about 10% to about 30%, or from about 15% to about 25%, of the total population placental particles being greater than about 150 um.

The composition of claim 2, wherein the PP composition has the following particle size distribution: from about 50% to about 80% of the placental particles being between about 5 um and about 80 um, from about 10% to about 30% of the placental particles being from about 80 um to about 150 um, and from about 10% to about 30% of the total population placental particles being greater than about 150 um, wherein the aforesaid percentages are percentages of a total population of placental particles present in the PP composition.

In an exemplary embodiment, the dry weight percentages of each of the APs, CPs, UCPs, and CPPs present in the PP composition are: from about 5 wt % to about 95 wt % APs, from about 5 wt % to about 95 wt % CPs, from about 0 wt % to about 90 wt % UCPs, and from about 0 wt % to about 90 wt % CPPs, based on the total dry weight of the PP composition.

In another exemplary embodiment, the dry weight percentages of each of the APs, CPs, UCPs, and CPPs present in the PP composition are: from about 10 wt % to about 30 wt % of APs, from about 30 wt % to about 75 wt % of CPs, from about 5 wt % to about 50 wt % UCPs, and from about 5 wt % to about 50 wt % CCPs,, based on the total dry weight of the PP composition.

In an exemplary embodiment in which the PP composition comprises at least APs, CPs, and either UCPs or CPPs, and the dry weight percentages of each of the APs, CPs, and either UCPs or CPPs present in the PP composition are: about 10-30% by weight (wt %) of APs, about 35-75 wt % of CPs, and about 15-45 wt % of UCPs or CPPs, based on the total weight of the PP composition.

In an exemplary embodiment in which the PP composition comprises APs, CPs, UCPs, and CPPs, and the dry weight percentages of each of the APs, CPs, UCPs, and CPPs in the PP composition are: about 10-30 wt % APs, about 10-50 wt % CPs, about 10-50 wt % UCPs and about 10-50 wt % CPPs, based on the total weight of the PP composition.

A method for producing placental particles and mixtures thereof, from a placenta which includes at least amnion, chorion, and umbilical cord, is also provided and comprises the steps of:
(A) separating one or more of the amnion, the chorion, and the umbilical cord from each other;
(B) cleaning each of the separated amnion and chorion, separately and lightly to remove loose blood clots from each placental tissue without causing damage to the tissues;
(C) cutting and cleaning the umbilical cord to facilitate contacting the umbilical cord with processing agents and removing additional blood clots and blood from the umbilical cord;
(D) decellularizing one or more of the amnion, chorion, and umbilical cord, separately from each other;
(E) optionally, freezing one or more of the decellularized amnion, chorion, and umbilical cord, and thawing each when processing is re-commenced;
(F) optionally, disinfecting one or more of the amnion, chorion, and umbilical cord, separately or together;
(G) cutting each of the one or more amnion, chorion, and umbilical cord into pieces having sizes suitable for milling in a milling apparatus;
(H) dehydrating each of the one or more of amnion, chorion, and umbilical cord, separately from each other, to produce dry amnion (AM) chips, dry chorion (CM) chips, and dry umbilical (UC) cord chips;
(I) optionally, storing each of the dry AM chips, the dry CM chips, and the dry UC chips, separately from each other, in sealed foil pouches until processing is continued;
(J) pre-milling the dry UC chips to form intermediate sized pre-milled UC chips which are smaller than the dry UC chips forming during the dehydrating step (H)
(K) combining together desired quantities of each of the dry AM chips, the dry CM chips, and the pre-milled UC chips, to produce a placental tissue mixture comprising predetermined proportions of each of the dry AM, CM, and UC chips in the mixture;
(L) milling the placental tissue mixture of dry AM, CM, and UC chips and pieces to produce a placental particle composition comprising a mixture of dehydrated amnion derived particles (APs), chorion derived particles (CPs), and umbilical cord derived particles (UCPs), wherein the placental particle composition comprises up to about 30% of placental particles having particle size greater than 150 um.

In an exemplary embodiment, the intermediate size of the pre-milled dry US chips produced by the pre-milling step (J) is smaller than the dry UC chips produced during dehydrating step (H) and about equal to or larger than sizes of the dry amnion (AM) chips and dry chorion (CM) chips produced during the dehydrating step (H).

In another exemplary embodiment in which the placenta further includes a placental disk having the chorionic plate lying thereon, the step of (A) separating the chorionic plate includes scraping edges of the placental disk on a maternal facing surface of the chorionic plate, scraping a side of the placental disk opposite the chorionic plate to further expose the chorionic plate and chorionic villi extending therefrom, and cutting away the chorionic villi, and placing the chorionic plate into water; the step of (B) cleaning includes cleaning the separated chorionic plate, separately and lightly to remove loose blood clots without causing damage to the tissues; the step of (D) decellularizing includes decellularizing the chorionic plate; the step of (G) cutting includes cutting the chorionic plate into pieces having sizes suitable for milling in a milling apparatus, the step of (H) dehydrating includes dehydrating the chorionic plate to produce dry chorionic plate (CP) chips; the step of (K) combining includes also combining a desired quantity of the dry CP chips to produce a mixture comprising predetermined proportions of each of the dry AM, CM, UC, and CP chips and pieces, respectively, in the mixture; and the step of (L) milling the mixture produces a placental particle composition comprising a mixture of dehydrated APs, CPs, UCPs and chorionic plate particles (CPPs).

Furthermore, the step of (J) pre-milling may comprise combining the dry UC chips and the dry CP chips to form mixture of dry UC chips and dry CP chips, and pre-milling the mixture to produce a mixture of intermediate sized pre-milled UC chips and intermediate sized pre-milled CP chips, which are smaller than the dry UC and CP chips formed during the dehydrating step (H); and the step of (K) combining includes combining desired quantities of each of the dry AM chips and the dry CM chips to the mixture of pre-milled UC and CP chips, to produce a mixture comprising predetermined proportions of each of the dry AM, CM, UC, and CP chips in the mixture.

### BRIEF DESCRIPTION OF THE FIGURES

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIG. 1 is a schematic diagram showing the chorionic plate of a placenta relative to other components.

FIG. 2A, 2B, 2C provide graphs showing the particle size distribution determined for several embodiments of the PP composition described and contemplated herein including, respectively, a 2-component PP composition (amnion, chorion), a 3-component PP composition (amnion, chorion, umbilical cord), and a 4-component PP composition (amnion, chorion, umbilical cord, chorionic plate);

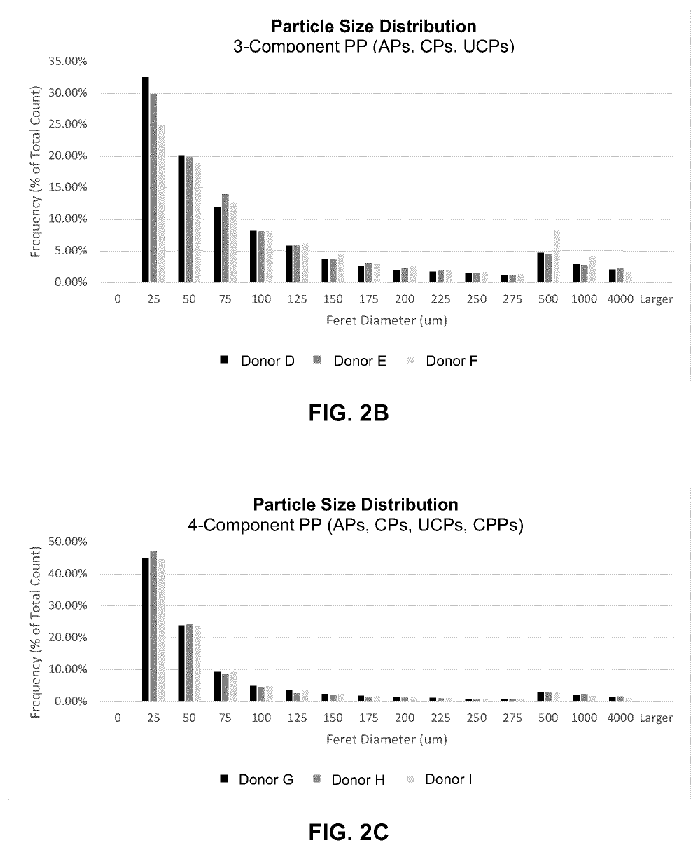

FIG. 3 is a graph showing relative enzyme degradation as measured by determination of material loss by a prior art placental particulate composition, PTP Composition, compared to a 2-component PP Composition (AM, CM, UC), and a 4-component PP Composition (AM, CM, UC, CP); and

FIG. 4 is a graph showing relative washout resistance as measured by determination of material loss after full hydration and exposure to several water washes of a prior art placental particulate composition, PTP Composition, compared to a 4-component PP Composition (AM, CM, UC, CP).

### DETAILED DESCRIPTION

The invention described and contemplated herein provides placental particle (PP) compositions which comprise a mixture of dehydrated placental particles derived from one or more types of placental tissue, including without limitation, amnion membrane, chorionic membrane, umbilical cord, and chorionic plate, any of which may be recovered from one or more placentas, In other words, some embodiments of the PP composition comprise a mixture of one or more of: amnion membrane derived particles (APs), chorionic membrane derived particles (CPs), umbilical cord derived particles (UCPs), and chorionic plate derived particles (CPPs).

In some exemplary embodiments, the PP composition comprises a mixture of APs and CPs, while other embodiments comprise a mixture of APs, CP, and either UCPs or CPPs. In some embodiments, the PP composition comprises a mixture of APs, CPs, UCPs and CPPs. In some embodiments, the PP composition comprises a mixture of UCPs and either APs, CPs, or both APs and CPs. In some embodiments, the PP composition comprises a mixture of CPPs and either APs, CPs, or both APs and CPs. In some exemplary embodiments, the PP compositions comprise a mixture of UCPs and CPPs, or a mixture of APs and UCPs, with or without CPPs, or a mixture of CPs and UCPs, with or without CPPs, It should be understood that all of the different types of particles present in a PP composition may be, but are not required to be, obtained or provided from the same placenta. Furthermore, the PP compositions may also, but do not have to, include one or more other tissue types or components (e.g., Wharton's jelly, placental disk, etc.) from one or more placentas,

The placental particles in the mixture have a particle size of from about 5 microns (um) to about 5800 um. Regardless of which of the one or more types of placental tissue are present in the PP composition, the PP composition has a particle size distribution of;
from about 60% to about 65% of the placental particles being between about 5 um and about 80 um,
from about 15% to about 20% of the placental particles being between about 80 um and about 150 um, and
from about 15% to about 25% of the placental particles being greater than
about 150 um,
wherein the aforesaid percentages are percentages of a total population of placental particles present in the PP composition.

When PP compositions comprising mixtures of placental particles as described above and having the aforesaid particle size distribution are applied or implanted as a wound covering. better (i.e., more thorough and complete) wound coverage is accomplished, with or without a dressing placed over and on top of the wound covering.

The invention described and contemplated herein also provides kits including one or more PP compositions, with or without one or more pharmaceutically acceptable excipients, carriers, or both. Methods of making the placental particles and the PP compositions having the aforesaid particle size distribution are also provided and described. Upon rehydration, the PP compositions with the aforesaid particle size distribution have a putty-like consistency which is cohesive, shapeable and reshapeable, and have handling characteristics which provide improved ease of application to a wound site, reduced loss from the site due to excess fluid/irrigation, and increased retention time at a wound site after a dressing is placed on top.

The PP compositions comprising mixtures of dehydrated placental particles having the aforesaid particle size distribution, as described and contemplated herein, are useful for treating internal and external tissue defects, such as by forming or being a protective covering or barrier during repair and reconstruction of, as well as promoting and even enhancing the healing process during such repair and reconstruction. For example, without limitation, internal and external tissue defects which are effectively treatable with the PP compositions include acute, chronic, and surgically created wounds. Examples of such wounds include, but are not limited to, partial and full-thickness wounds, pressure ulcers, venous ulcers, diabetic ulcers, chronic vascular ulcers, tunneled or undermined wounds, surgically created wounds (such as autograft donor sites), dehisced wounds, trauma wounds (abrasions, lacerations, and skin tears), draining wounds, partial-thickness burns, and combinations thereof.

Furthermore, the invention described and contemplated herein provides methods for treating various musculoskeletal disorders and other conditions using such PP compositions, including osteoarthritis (OA), degenerative disc disease, tendonitis, plantar fasciitis, and pain associated therewith. Methods are also provided for prophylactic and cosmetic treatments.

As used herein, the term “about” encompasses the explicitly recited amounts as well as deviations therefrom of ±10% of such explicitly recited amounts.

The terms “administer” and “apply,” in all their grammatical forms, refer to placing, delivering, depositing, injecting, implanting, layering, spreading, etc., a quantity of a substance or material on, in, adjacent to, or a combination thereof, a wound or otherwise damaged or injured tissue (i.e., host tissue) which is expected to benefit from such administration.

The term “biologically compatible liquid or gel” includes any diluent, carrier, etc., including without limitation a suitable solution, buffer, or excipient, preferably at point of care. Exemplary solutions include but are not limited to normal saline (0.9% sodium chloride), a physiological salt solution (phosphate buffered saline; PBS), Dulbecco's Modified Eagle Solution (DMEM), water, fibrin glue, antibiotics, whole blood, any autologous preparation (such as platelet rich plasma (PRP), bone marrow aspirate concentrate (BMAC), stromal vascular fraction (SVF)), corticosteroid, a solution containing hyaluronic acid (HA) or anti-inflammatory agents, and balanced salt solution (BSS).

The terms “dehydrating” and “dehydrated” refer respectively to removal of at least a portion (i.e., a portion or substantially all) of water present in tissue, material, and compositions comprising one or both, and the condition of at least a portion of water present having been removed therefrom. Dehydrating may be performed by any of several techniques including, but not limited to, heating, air drying, desiccation, lyophilizing, and combinations.

The term “derived” is used herein to describe circumstances in which a material or substance has been made from an original or intermediate material, tissue, or substance, for example, without limitation, through physical processing, chemical processing, or a combination thereof. The aforesaid processing may involve one, two, or even several steps or phases. For example, as described below, amnion derived particles are derived from amnion membrane which may be subjected to one or more processing steps such as separation from chorion membrane and other placental components, size reduction, decellularization, rinsing, disinfection, more rinsing, dehydration, and mixing with other types of placental particles.

The term “diluent” refers to chemical compounds that are used to dilute the compound or composition of interest prior to delivery. Salts dissolved in buffered solutions (which also can provide pH control or maintenance) are utilized as diluents in the art, including, but not limited to a phosphate buffered saline solution and sodium chloride solutions.

The terms “hydrate” and “rehydrate,” in all their grammatical forms, mean to add a biologically compatible liquid or gel, e.g., a diluent or carrier, to a material to provide a more malleable or flowable mixture comprising the material which has handling characteristics enabling easier administration or application of the mixture, whether manually, using an instrument such as a spatula, or passing the mixture through a cannula, syringe, or needle (i.e., injecting). Insufficient hydration or rehydration occurs when not enough liquid or gel has been added to a material for the resulting mixture to be administered or applied by the preferred method (e.g., passing through a cannula or injection through a syringe or needle, etc.). Overhydration occurs when the quantity of liquid or gel added to a material forms a mixture that lacks sufficient cohesiveness for effective and controlled administration or application by the preferred method (e.g., manual shaping or reshaping and placement, deposition and spreading using a spatula or other instrument, passing and controlled deposition through a cannula or syringe, etc.).

The terms “lyophilizing” and “lyophilized” refer to the process of freeze drying which includes a freezing phase and one or more drying phases, and the condition of having been subjected to a lyophilizing process. Lyophilizing often enables or prolongs the preservation of a tissue, material, or substance, for a period of time longer than without lyophilizing and with storage at temperatures above freezing (e.g., above 0° C.).

The term “pharmaceutically acceptable,” as used herein, refers to a material which is relatively nontoxic, i.e., the material may be administered to an individual without causing undue undesirable biological effects or interacting in a deleterious manner with any of the components of the composition in which it is contained.

The term “placental tissue” as used herein refers to whole placenta and components of thereof, including that which has been modified by cleaning and separating the various components and types of tissues, such as but not limited to, amnion membrane, chorionic membrane, umbilical cord, placental disk, and other placental components and types of tissue. Furthermore, placental tissue may include extracellular matrix layers naturally found in the placenta, such as one or more of: an epithelial layer, a fibroblast layer, a trophoblast layer, an intermediate (or “spongy”) layer, Wharton's jelly, etc.

In many mammals, including but not limited to humans, the placenta surrounds the fetus during gestation within the mother's uterus and comprises several tissue types including, but not limited to, amnion, chorion, and umbilical cord. The amnion, or amniotic membrane (AM), is the innermost layer of the placenta, closest to the fetus and separating the mother from the fetus throughout the baby's development. More particularly, the amniotic membrane forms an avascular membranous sac which is filled with amniotic fluid to contain and support the fetus therein.

The chorion, or chorionic membrane (CM), surrounds and is substantially coextensive with the amniotic membrane, thereby forming the placental sac, with the chorionic membrane as the outer layer. The chorionic membrane separates the amniotic membrane from the uterine wall, but is itself in contact with the uterine wall to allow the exchange of nutrients, oxygen and waste products with the mother. An intermediate or “spongy” layer fills space between the amniotic and chorionic membranes and allows these membranes to slide against one another.

The umbilical cord (UC) provides a pathway for the exchange of oxygenated, nutrient-rich blood from the mother to the fetus and low-oxygen, nutrient-depleted blood from the fetus to the mother. More particularly, in humans and some other mammals, the umbilical cord is a tube filled with Wharton's jelly and has two arteries and one vein extending between the fetus and the chorionic membrane.

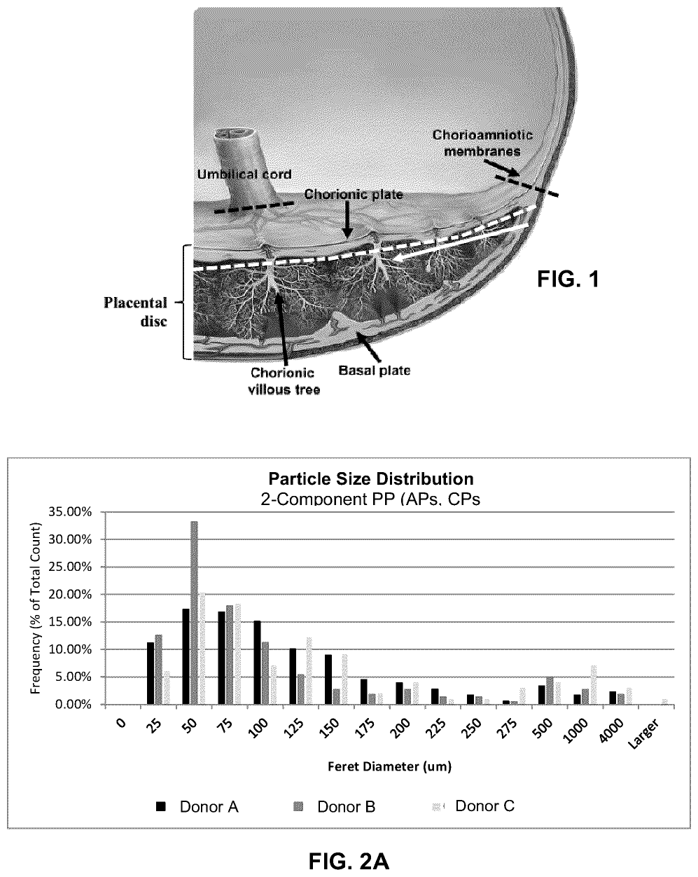

The chorionic plate (CP) is described with reference to FIG. 1 which provides a schematic cut away view of a portion of a placenta and the associated umbilical cord that has been adapted from the colorized version provided as FIG. 1 in Kim, C.J., et al., Chronic inflammation of the placenta: definition, classification, pathogenesis, and clinical significance. Amer. J. Obstet. Gynecol. 2015; 213(4 Suppl): S53-S69. doi:10.1016/j.ajog.2015.08.041. As shown in present FIG. 1, the chorionic plate is a feature of the placenta which is generally understood to be a fetal (fetus-facing) surface of the placental disk and connects the blood supply of the developing fetus to the mother. The umbilical cord extends roughly through the middle or center of the chorionic plate, which is covered by the amnion. Isolation of the chorionic plate may, for example, be accomplished by: cutting and removing the umbilical cord, (see dotted line at base of umbilical cord in FIG. 1), peeling away the amnion from the chorion/placenta disk and cut from the placental disk, the chorion membrane is cut away, around the edge of the placental disc (see dotted line to the right denoting junction of placental disk and chorion membrane in FIG. 1), then scraping away (see direction and location of arrow in FIG. 1) softer tissues and basal plate from side of the chorionic plate opposite its fetal side (see dotted line extending adjacent to the chorionic plate in FIG. 1), and finally cutting away chorionic villi much like pruning “roots”.

The term “point of care” is used herein to mean at or near the point in time when a clinician or other health care provider administers health care services and/or products, including the composition of the invention, to a patient.

The terms “resuspend” or “resuspended” are used herein to refer to the addition of a biologically compatible liquid or gel, e.g., a diluent or carrier, to a dehydrated or insufficiently hydrated material, to provide a flowable mixture comprising the material and that possesses the desired consistency and handling characteristics for administration or application by a preferred method, most often by passing the mixture through a cannula, syringe, or needle (i.e., injecting).

The term “therapeutically acceptable” with respect to a formulation, composition or component, as used herein, means having no persistent detrimental effect on the general health of the subject being treated.

The term “therapeutically effective amount,” as used herein, refers to a sufficient amount of an agent or a compound or composition being administered which will relieve, partially or fully, one or more of the symptoms of the disease or condition being treated, e.g., tissue damage or associated pain or other symptoms or causes of the treated disease.

All publications mentioned herein are incorporated by reference in their entirety. In the case of conflict, the present specification, including definitions shall control. In addition, the particular embodiments discussed below are illustrative only and not intended to be limiting.

Generally, the characteristics of individual particles in any population of particles is determinable by any of several microscopy techniques or other measurement techniques known to those of ordinary skill in the relevant art. Based on the particle characteristics (e.g., particle counts and statistics of each counted particle, such as area and Feret Diameter (i.e., caliper diameter or widest diameter across a given particle) which are measured for a population of particles, statistical analysis may be applied to determine several other quantifiable characteristics of the particles and the population of particles including, but not limited to, minimum, mean, average, and maximum particle sizes, particle size distribution (e.g., distribution among defined data bins of different size ranges), which may alternatively be expressed as particle size percentiles, and other characteristics of the particles and the population of those particles.

For purposes of this disclosure, the method for determining the particle characteristics of individual particles in the PP compositions described and contemplated herein, as well as particle size characteristics of the PP compositions, involves performing multiple field microscopic imaging of a quantity of the population of particles spread, single layer, across a microscopic glass slide, followed by processing using software, such as ImageJ (which is a Java-based image processing program developed at the United States National Institutes of Health and the Laboratory for Optical and Computational Instrumentation (LOCI, University of Wisconsin, U.S.A.)) to analyze the images via the Analyze Particles function, producing a table of particle characteristics, using the Feret Diameter parameter and then statistical analysis of the particle characteristics data, with software such as Microsoft Excel, to determine the particle size properties of the PP composition. Optionally, clear tape may be used to aid in transferring and/or immobilizing the dry dispersed particles for analysis.

If counting by field rather than a single image, the counted particles per field can be combined to yield a full list of all counted particles and their sizes for the entire imaged slide.

Statistics using software such as Microsoft Excel can then be analyzed to generate characteristics statistics such as minimum, mean, average, and maximum particle size, particle size distribution, and distribution of particle among defined data bins of different size ranges (e.g., percentiles).

As will be described in more detail below, in one aspect of the invention, there are provided placental particles that are prepared by processing, preferably separately from one another, one or more types or components of placental tissue including, but not limited to, amniotic membrane (AM), chorionic membrane (CM), umbilical cord (UC), and chorionic plate (CP) components of one or more human placentas. Each of the aforesaid components may be obtained from a single donor placenta and processed separately. However, it is also possible to obtain the placenta components from two or more placentas obtained from different donors.

More specifically, AM derived particles (APs) are placental particles derived from an AM component of a placenta. CM derived particles (CPs) are placental particles derived from a CM component of a placenta. UC derived particles (UCPs) are placental particles derived from a UC component of a placenta. CP derived particles (CPPs) are placental particles derived from a CP component. Each of the APs, CPs, UCPs, and CPPs may, independently of one another, be dehydrated (which means partially or substantially completely dehydrated). Furthermore, each of the APs, CPs, UCPs, and CPPs may, independently of one another, have the same or different water content (i.e., be dehydrated or rehydrated to the same or different degrees). In some embodiments, the APs, CPs, UCPs, and CPPs are all dehydrated and have about the same water content as one another.

In some embodiments, the PP compositions disclosed and contemplated herein each comprise a mixture of dehydrated placental particles comprising from about 0 weight % (wt%) to about 100 wt % APs, about 0 wt % to about 100 wt % CPs, about 0 wt % to about 100 wt % UCPs, and about 0 wt % to about 100 wt % CPPs, based on the total weight of the placental particles present in the PP composition. While the PP compositions must include either both APs and CPs, or all three of the aforesaid APs, CPs, and UCPs, the PP compositions may also include material derived from one or more other types of placental tissues. In some embodiments, the PP compositions disclosed and contemplated herein each comprise a mixture of dehydrated placental particles comprising from about 5 wt % to about 95 wt % APs, about 5 wt % to about 95 wt % CPs, 5 wt % to about 95 wt % UCPs, and about 5 wt % to about 95 wt % CPPs, based on the total weight of the placental particles present in the PP composition. In some embodiments, the PP compositions disclosed and contemplated herein each comprise a mixture of dehydrated placental particles comprising from about 5 weight % (wt%) to about 85 wt % APs, about 5 wt % to about 80 wt % CPs, 5 wt % to about 85 wt % UCPs, and about 5 wt % to about 85 wt % CPPs, based on the total weight of the placental particles present in the PP composition. While the PP compositions will include at least one of APs, CPs, UCPs, CPPs, the PP compositions may also include material derived from one or more other types of placental tissues.

In some embodiments, the proportions of APs, CPs, UCPs, and CPPs present in the PP composition may be, but are not required to be, determined by the proportions of AM, CM, UC, and CP present in the placenta from which the three types of placental particles are produced. In other embodiments, the proportions of APs, CPs, UCPs, and CPPs present in the PP composition may be, but are not required to be, determined by the properties it is desired for the resulting PP composition to have.

Additional materials may also be included in PP compositions containing placental particles in a particle size distribution as described above. As will be readily recognized, such PP compositions combined with one or more additional materials may produce a wide range of grafts adapted and designed for particular or specialized applications and treatments. Non-limiting examples of such additional materials include for example, without limitation, one or more tissue-derived matrices, hyaluronic acid (HA) and derivatives thereof, antibiotics, anti-inflammatories, corticosteroids, surfactants, anti-caking agents, natural and synthetic polymers, exogenous growth factors and cytokines, excipients, carriers, hydration fluids (including without limitation water, saline, phosphate buffered saline (PBS), blood plasma, HA and its derivatives, and the like.

The physical or geometric form of the one or more tissue-derived matrices included as an additional material in the PP compositions is not particularly limited and may, for example without limitation, include particulates, flakes, pieces, slivers, fibers, elongated pieces, chunks, sheets, irregular shapes, and combinations thereof. The type of tissue from which the tissue-derived matrices are produced is not particularly limited and may include, for example without limitation, dermis, adipose, fascia, bone, muscle, cartilage, tendon, ligament, nerve, and combinations thereof.

Examples of natural polymers suitable for inclusion as additional materials in the PP compositions include, without limitation, collagen, gelatin, fibrinogen, hyaluronan, silk, agarose, chitosan, and combinations thereof. Examples of synthetic polymers include, without limitation, poly (lactic-co-glycolic acid) (PLGA), polylactic acid (PLA), polyglycolide (PGA), polyurethane (PU), polycaprolactone (PCL), and polyethylene glycol (PEG), and combinations thereof.

Each of the types of placental particles described herein is prepared from mammalian placenta, such as, but not limited to, human placenta, whether delivered by Cesarean section or by natural childbirth. The placenta may be processed immediately (e.g., within about 24 hours) after recovery, or stored at about 2° to 8° C. (i.e., refrigeration temperatures) for example for up to 120 hours post-recovery, preferably no longer than 72 hours post-recovery. It has been have found that freshly obtained placenta (stored no longer than 120 hours at 2° to 8° C., for example, preferably no longer than 72 hours post-recovery) significantly reduces the risk of degeneration of the recovered placental tissues. However, frozen AM, CM, UC, CP, and other types of placental tissue may be used, in place of fresh tissue or in combination with fresh tissue.

Generally, the use of placental tissue in any physical form for treating a wound or otherwise damaged or injured tissue (i.e., host tissue) involves administering a quantity of the selected placental tissue form to the host tissue. Different physical forms of placental tissue have different advantages and disadvantages, as follows.

For example, a sheet (or patch) of placental tissue has a first surface and an opposite second surface, both defined by lengths and widths of at least about 1 centimeter each, and has a thickness between the first and second surfaces. Due to this configuration, a sheet of placental tissue generally maximizes the area of contact between the placental tissue and the host tissue being treated, and provides a longer time period of such contact (i.e., a reduced degradation rate or remodeling rate), as compared to particulate forms of placental tissue. Reduced degradation rate or reduced remodeling rate of any tissue form provides a prolonged presence of the tissue form, which is believed to provide prolonged or extended period of biologic activity (e.g., enhanced wound healing, reduced inflammation, etc.). A placental tissue sheet so administered also generally provides more continuous coverage of the host tissue, as compared to particulate placental tissue forms, which reduces infiltration and through flow by fluid (whether host fluid or added hydration fluid).

Sheets of placental tissue also maintain contact between the placental tissue and the host tissue better than most particulate placental tissue forms, at least in part because the sheets have a greater resistance to displacement by hydration and over hydration (by either host fluid or added fluid). A disadvantage of sheet forms of placental tissue is their reduced capability to conform to and fully contact host tissue having an irregular surface area, i.e., having an irregular perimeter (e.g., other than circular, oval or rectilinear) or having portions with different or highly variable depths (e.g., both relatively superficial and deep), tunneling, or highly textured (e.g., unevenly disintegrated).

Particulate forms of placental tissue, on the other hand, generally have a greater capability than sheet forms to conform to, contact and cover host tissue having an irregular surface area as described above. However, particulate placental tissue is more susceptible (i.e., less resistant) to disturbance, or even displacement of the placental tissue from portions, or all, of the host tissue to be treated. Disturbance of particulate placental tissue forms, after administration or placement at a treatment site, generally means unintentional washing away of a portion of the placental tissue, which results in areas of variable thickness or depth across the host tissue being treated (e.g., treatment site). Displacement of particulate placental tissue forms, after administration or placement at a treatment site, generally means complete removal (i.e., 95 wt %) of the tissue form from the treatment site.

On the other hand, the PP compositions described and contemplated herein, retain benefits and improvements of both the particulate and sheet forms, while reducing or avoiding some disadvantages or otherwise less preferred features. The PP compositions comprise a mixture of placental particles as described above,, wherein the placental particles in the mixture have a particle size of from about 5 microns (um) to about 5800 um, and the PP compositions have a particle size distribution of:
from about 60% to about 65% of the placental particles being between about 5 um and about 80 um,
from about 15% to about 20% of the placental particles being between about 80 um and about 150 um, and
from about 15% to about 25% of the placental particles being greater than about 150 um,
wherein the aforesaid percentages are percentages of a total population of placental particles present in the PP composition.

When the PP composition is hydrated (or rehydrated), a PP composition putty is formed which is shapeable and reshapeable and also more cohesive and more displacement resistant than a hydrated mass or population of placental tissue particulates having a particle size range only up to about 150 um, or a particle distribution wherein less than about 15% of the placental particles are greater than about 150 um, or both. The PP compositions also tend to be more shapeable and reshapeable than placental tissue sheets, whether or not hydrated or rehydrated.

More particularly, compared to known particulate placental tissue forms, when administered to host tissue, the PP compositions provide one or more of the following characteristics and properties: (1) a greater total area of contact between the placental tissue and the host tissue being treated (i.e., better or more complete coverage of host tissue, especially wounds having irregular or deep configurations), (2) a reduced degradation rate or remodeling rate (e.g., where degradation and remodeling occur due to enzymatic activity or other healing and reconstructive biological processes), (3) greater resistance to displacement by irrigation, hydration, and over hydration (by either host fluid or added fluid); and (4) greater resistance to over hydration (i.e., greater ability to maintain desired handling characteristics over a wider degree of hydration or rehydration). Furthermore, in contrast to sheet forms of placental tissue, when administered to host tissue, the PP compositions having the aforesaid particle size distribution provide greater or enhanced capability to conform to and cover host tissue having an irregular surface area as described above.

It is further noted that, when the PP compositions having the aforesaid particle size distribution are hydrated with a biocompatible fluid (e.g., a liquid or gel diluent, carrier, or combination thereof), a cohesive shapeable putty is formed that easily conforms to contours and irregularities in the geometry of host tissue to be treated and resists displacement by hydration and over hydration. The PP compositions having the aforesaid particle size distribution also retain handling characteristics better when overhydration (e.g., excess hydration or rehydration) occurs. The hydrated PP composition putty is more cohesive, more shapeable, and more displacement resistant than a hydrated mass or population of placental tissue particulates having a particle size range only up to about 150 um, or a particle distribution wherein less than about 15% of the placental particles are greater than about 150 um, or both. The PP compositions are also more reshapeable than placental tissue sheets, whether hydrated or not.

The combination of UCPs with APs and CPs in the PP compositions described and contemplated herein is believed to provide several benefits and advantages, as follows. For example, including UCPs in the composition with the APs and CPs more efficiently utilizes donated placentas and increases product yield from each placenta. Moreover, since the extracellular matrix of the UC tends to be more dense than AM or CM and still provides similar biological activity, UCPs are an excellent bulking agent in the PP compositions, without reducing or diluting the beneficial biological activity of the APs and CPs.

It is also believed, but without being limited by theory, that embodiments of the PP compositions which include the more dense UCPs may have a lower degradation rate and/or a lower remodeling rate after administration, as compared to compositions comprising only APs and CPs. Finally, including more dense UCPs in the PP composition may also increase migration resistance of the composition upon hydration, after administration. Reduced degradation rate and/or remodeling rate and increased migration resistance all serve to prolong contact of the PP composition with host tissue being treated which, in turn, extends or prolongs the period of biological activity as compared to such compositions without UCPs.

It has been found that each of the AM, CM, UC, and CP placental components contains beneficial endogenous growth factors and cytokines. In particular, each component has measurable levels of pro-anabolic (bFGF), anti-catabolic (TIMP-1,-2,-3), and anti-inflammatory (IL-1Ra) growth factors and cytokines; and consistently low levels of inflammatory factors (IL-1α). Without wishing to be bound by theory, the levels the aforesaid compounds are believed to be sufficiently comparable amongst the AM, CM, UC, and CP components such that a formulation using a component ratio that maximizes the product yield from the donor placenta is expected to be particularly beneficial, as a practical matter, since it tends to maximize efficient use of donated raw materials.

In certain embodiments, the PP composition comprising a mixture of placental particles, as described above, may be utilized in a dehydrated (dry) form, or combined with a sufficient quantity of biologically compatible fluid (e.g., diluent or carrier) to form a flowable suspension which is deliverable by syringe (i.e., deliverable through syringe tip, without a needle), or rehydrated with a biologically compatible fluid to form a putty.

As will now be explained in detail, the invention described and contemplated herein also provides methods for producing the dehydrated placental particles derived from one or more of the AM, CM, UC, and CP, as well as preparing PP compositions comprising mixtures of such dehydrated placental particles.

The production method described herein provides a PP composition having the particle size distribution described about and comprising a mixture of APs, CPs, and optionally UCPs, CPPs, or both UCPs and CPPs having the particle size range described above. The PP compositions are capable of delivery (administration) via syringe (i.e., injectable, whether dried or not and deliverable through syringe tip, without a needle), in dry form, or as a putty. Furthermore, the production method involves processing procedures that do not adversely affect growth factors and cytokines of interest which are intrinsic to the placental tissue. When the PP composition is provided in dried form, it may or may not be combined with a sufficient quantity of biocompatible liquid to produce an injectable composition or a putty, prior to or at the time of use or implantation. The entire method for preparing the placental particles and PP compositions comprising mixtures of them may be performed aseptically.

The method for producing the placental particles and mixtures thereof, as described and contemplated herein, from a placenta which includes at least amnion, chorion, umbilical cord, and chorionic plate components, generally comprises the steps of:
(A) separating one or more of the amnion, chorion, umbilical cord, and chorionic plate components from each other and the placental disc;
(B) cleaning each of the amnion and chorion, separately and lightly to remove loose blood clots from each placental tissue without causing damage to the tissues;
(C) cutting and cleaning the umbilical cord, which increases exposure of the umbilical cord for contacting with processing agents and facilitates removing additional blood clots and blood from the umbilical cord;
(D) decellularizing each of the amnion, chorion, and umbilical cord separately;
(E) optionally, freezing each of the decellularized amnion, chorion, and umbilical cord, for storage until processing is continued (i.e., a first “staging” step), and thawing each when processing is re-commenced;
(F) optionally, disinfecting each of the amnion, chorion, and umbilical cord, separately or together;
(G) cutting each of the three tissues into pieces suitable for milling in a milling apparatus,
(H) dehydrating each of the three tissues separately to produce three tissue types (i.e., AM, CM, and UC) of dry tissue “chips;”
(I) optionally, storing each of the dry AM, CM, and UC tissue “chips” in sealed foil pouches until processing is continued (i.e., a second “staging” step);
(J) pre-milling the umbilical cord chips to form intermediate sized pieces or particles of umbilical cord (e.g., using a hand mill, freezer mill, etc.) which will ultimately yield dimensions comparable to those of the milled AM pieces (chips) and CM pieces (chips) produced in milling step (L);
(K) combining together desired quantities of each of the dehydrated amnion chips, chorion chips, and umbilical cord pieces (which have been pre-milled) to produce a mixture comprising predetermined proportions of each placental tissue in the mixture;
(L) milling the combined dehydrated amnion, chorion, and umbilical cord pieces and particles to produce a placental particle composition comprising a mixture of dehydrated amnion derived particles (APs), chorion derived particles (CPs), and umbilical cord derived particles (UCPs); and
(M) optionally, sterilizing the composition, such as, without limitation, by exposing the composition to gamma radiation, e-beam radiation, UV light, ethylene oxide, etc.

It should be understood that the labels (e.g., (A), (B), (C) . . . ) of the foregoing steps of the presently described and contemplated method for producing APs, CPs, and UCPs, as well as the PP composition comprising a mixture thereof, do not indicate any required order of performing those steps. Rather, the labels of the method steps listed above provide a nomenclature which facilitates further discussion of each method step. Additionally, the labels of the foregoing method steps provide an exemplary recommended sequence of performing those steps, which may be modified as is within the capability and according to the preference of persons of ordinary skill in the relevant art.

In some embodiments, the processing method further comprises packaging desired quantities of the PP composition. For example, desired quantities of the PP composition may be measured and placed into containers. For example, without limitation, desired quantities of from 40 to 1500 milligrams (mg) of the PP composition may be measured and placed into glass vials or other containers. The containers (e.g., glass vials, etc.) may then be closed and sealed, for example without limitation, under vacuum.

In some embodiments, an additional step of dehydrating the PP composition may be performed. For example, without limitation, when it is desired to reduce final residual moisture of the PP composition, after measured quantities of the PP composition have been placed into containers, a second lyophilizing process may be performed, prior to closing and sealing the containers.

In some embodiments, the step of (C) cutting and cleaning the umbilical cord is performed by cutting the umbilical cord lengthwise (“butterfly”) and scoring it with a scalpel to expose the lumen and blood vessels therein, and lay the umbilical cord relatively flat for further processing. This cutting and cleaning step (C) increases exposure of the umbilical cord for contacting with processing agents and facilitates removing additional blood clots and blood from the umbilical cord. In some embodiments, the blood vessels in the umbilical cord may, but are not required to be disrupted and removed, e.g., by scraping.

In some embodiments, the step of (D) decellularizing each of the amnion, chorion, and umbilical cord separately is performed by contacting each with a decellularizing solution, followed rinsing each tissue with water one or more times to remove excess decellularizing solution. The decellularizing solution may, for example without limitation, comprise 1N NaCl.

In some embodiments, the step of (F) disinfecting is performed by contacting each tissue with a disinfection solution, followed by one or more (e.g., 2. 4, 6, 8, etc.) rinsing steps with water to remove excess disinfecting solution. Optionally, (F) disinfecting may be performed with two or more of the amnion, chorion, and umbilical cord tissues in the same vessel. After the step of (F) disinfecting, all three of amnion, chorion, and umbilical cord placental tissues have been cleaned of remaining blood components that may have elicited an immunogenic response.

In some embodiments, the step of (G) cutting each of the three tissues into pieces suitable for milling in a milling apparatus includes folding each of the amnion and the chorion folded into approximately 4×4cm pieces. In some embodiments, the step of (G) cutting each of the three tissues into pieces includes cutting the umbilical cord cut into 4 cm lengths and, if needed, performing further scoring enable laying the umbilical cord substantially flat.

In some embodiments, the step of (H) dehydrating each of the three tissues separately is performed by lyophilizing each tissue type to produce the three tissue types of dry tissue “chips.” After dehydrating each of the dry amnion, chorion, and umbilical cord chips may be weighed. Weighing at this stage enables determination of the available quantities of each placental tissue type available for combining and mixing to produce the PP composition having selected proportions of each of the APs, CPs, and UCPs.

The step of (J) pre-milling the umbilical cord is advantageous due to the larger, denser pieces of umbilical cord initially recovered and produced from the placenta. Pre-milling the umbilical cord (step J) provides intermediate pieces or particles of umbilical cord, which, in turn, after combining and milling the three placental tissue chips together, produces a more homogenously sized and shaped mixture of APs, CPs, and UCPs for the PP composition. In the absence of performing the step of (J) pre-milling the umbilical cord, combining and milling the AM, CM, and UC chips together produces a mixture which contains noticeably larger pieces of UC, as compared to the resulting APs and CPs.

The pre-milling performed in step (J) (i.e., pre-milling the umbilical cord), as well as the milling performed in step (L) (i.e., milling the combined dehydrated amnion, chorion, and pre-milled umbilical cord pieces), may each be performed by any technique and using any apparatus capable of controllably reducing chips one or more of dehydrated amnion, chorion, umbilical cord, and combinations of two or more thereof, to smaller particles having desired particle sizes and particle size distribution, as described above and contemplated herein. The (J) pre-milling and (L) milling may be performed, for example, without limitation, using any one or more manual, pneumatic, or electrically powered devices such as: a hand mill, analytical mill, cutting mill, a freezer mill, a grinding mill, a high energy mixer mill, a ball mill, and ring and puck mill, etc.

The step of (J) pre-milling umbilical cord is performed, at least in part, to produce intermediate sized pieces or particles of umbilical cord, which are then combined together with the AM chips and CM chips in a mixture. The step of (L) milling resulting the mixture is performed, at least in part, to produce the placental particle (PP) composition comprising a mixture of dehydrated amnion derived particles (APs), chorion derived particles (CPs), and umbilical cord derived particles (UCPs), having the aforesaid particle size distribution. The PP composition has one or more properties including, but not limited to, improved cohesiveness, reduced degradation rate, and reduced resorption rate compared to other placental derived particulate compositions, which lack umbilical cord derived particles, have a different particle sized range, have a different particle size distribution, or a combination of these properties.

Further details, exemplary embodiments, and options for the method of producing APs, CPs, and UCPs, and compositions comprising a mixture thereof will now be provided and explained.

In some embodiments, the APs, CPs and UCPs are derived from fresh placental tissue. In some embodiments, the APs, CPs and UCPs are derived from placental tissue which has been refrigerated or frozen for a period of time.

The method for preparing the placenta particles may involve first detaching the UC from the AM and CM components of the placenta, and also separating the AM and CM from one another. Cleaning of the AM, CM, and UC is also performed, in which blood clots, if present, are removed from the AM and CM, and blood vessels are removed from the UC.

In some embodiments of the method, all extracellular layers of the amnion, chorion and umbilical cord present in native tissue are retained during processing. In some embodiments, one or more or more layers of one or more of the AM, CM, and UC (for example but not limited to, one or more of the epithelial layer, spongy layer, trophoblast layer, Wharton's Jelly, etc.) may be removed.

In some embodiments of the method, the AM, CM, and UC placental tissues are rinsed one or more times, for example in distilled water, phosphate buffered saline (PBS), or other biocompatible aqueous solutions, to remove blood and other unwanted substances and contaminants. Preferably, but not necessarily, the three tissue types are rinsed separately. By rinsing the tissues as separate components, contaminants and blood are more easily and thoroughly removed. Rinsing can be done at room temperature or under cooling using.

The three tissue types (i.e., AM, CM, and UC) may be each cut into smaller pieces, e.g., 4 cm strips for further processing. The pieces are dehydrated, e.g., lyophilized for about 12 to 20 hours, such as about 16 to 20 hours or using other method of dehydration. Following dehydration, the pieces are milled, optionally, with some or all of each tissue type milled separately. Any milling process that reduces the placental tissues to an appropriate particle size while maintaining the tissues in a dry state while milling may be used in the present invention.

Before milling, each of the tissues (i.e., AM, CM, and UC) is optionally weighed to determine the amount of each tissue to be included in the final PP composition. For example, weighing the separate AM, CM, and UC components would provide information regarding the relative yields of each tissue type from the one or more placentas processed to produce them. As another example, weighing the separate AM, CM, and UC components enables the opportunity to select desired predetermined quantities of each component for combination into the mixed composition to be produced. If milled separately, the milled placental tissues are recombined thereafter for further processing. In embodiments where the tissues are milled separately and combined only after all milling is completed to produce PP particulates having the desired particle sizes and distribution, instead of pre-milling the UC, the UC would likely need to be milled longer or at a higher speed (rpm) to produce UC particles of a size comparable to that of the AM and UC particulates. The size of the placental tissue particulates may be selected to ensure that most particles are of similar scale and of maximize size (for example, but not limited to, having few or no particles larger than about 0.5-1 cm).

The milled tissue mixture may be aliquoted by weight and optionally may be dehydrated a final time, e.g., lyophilized for 12 to 20 hours, such as 18 hours. The residual moisture content of the final dehydrated or lyophilized placental particles is preferably less than 15%, more preferably less than 6%.

The aliquots of lyophilized or otherwise dehydrated placental particles may be distributed in individual containers such as glass vials that are stoppered under vacuum and capped to create a hermetic seal, for example. The aliquoted, lyophilized tissue is, optionally, sterilized by E-Beam irradiation, gamma irradiation, UV light or exposure to ethylene oxide, supercritical carbon dioxide or other suitable sterilant known to those in the art. E-beam irradiation is a preferred method of sterilization. The dehydrated, sterilized PP compositions are substantially free of blood residuals and foreign matter.

In some embodiments, as stated previously, the PP compositions of the invention may comprise from about 5 wt % to about 95 wt % APs, about 5 wt % to about 95 wt % CPs, and about 0 wt % to about 95 wt % UCPs. In some embodiments, the PP compositions comprise a mixture of placental particles containing from about 10 wt % to about 30 wt % APs, from about 30 wt % to about 75 wt % CPs, and from about 5 wt % to about 60 wt % UCPs. Regardless of the proportions of the APs, CPs, and UCPs in the PP composition, the particle size distribution is as previously stated above.

In another aspect of the invention, the dehydrated placental particle composition can be rehydrated with a biologically compatible liquid or gel, such as without limitation, a suitable solution, buffer, or excipient, preferably at point of care. Exemplary solutions include but are not limited to normal saline (0.9% sodium chloride), a physiological salt solution (phosphate buffered saline; PBS), Dulbecco's Modified Eagle Solution (DMEM), water, any autologous preparation (such as platelet rich plasma (PRP), bone marrow aspirate concentrate (BMAC), stromal vascular fraction (SVF)), corticosteroid, a solution containing HA or anti-inflammatory agents, and balanced salt solution (BSS).

The ratio of rehydrating solution and PP composition can be varied as needed. In some procedures a more concentrated preparation is useful, whereas in other procedures, a combination of solution with a lower concentration of the placental particles may be more useful. In various embodiments of the invention, additional compounds or components can added to the composition. Exemplary compounds that can be added to the rehydrated formulation comprising a PP composition include but are not limited to pH modifiers, buffers, collagen, HA, anti-inflammatories, surfactants, stabilizers, proteins, and the like. Antimicrobial agents such as antibiotics or anti-fungal agents may be added. Other substances can be added to the compositions to stabilize and/or preserve the PP compositions if needed. The material can be packaged and stored, for example, at room temperature, under refrigeration, or for example, at −20° C. or −80° C. prior to use.

Pharmaceutical compositions may be formulated in a conventional manner using one or more physiologically acceptable carriers including excipients and auxiliaries which facilitate processing of the PP compositions into preparations which can be used pharmaceutically. Formulation is dependent upon the desired route of administration. Any of the well-known techniques, diluents, carriers, and excipients may be used as suitable and as understood in the art. A summary of pharmaceutical compositions described herein may be found, for example, in Remington: The Science and Practice of Pharmacy, Nineteenth Ed (Easton, Pa. Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins; 1999), herein incorporated by reference in their entirety.

In certain embodiments, the compositions include a pharmaceutically acceptable diluent(s), excipient(s), or carrier(s). In addition, resuspended placental particles and hydrated PP compositions described herein can be administered as pharmaceutical compositions in which the PP compositions are mixed with other active ingredients, as in combination therapy. In some embodiments, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

The PP compositions described and contemplated herein are useful in methods for treating a wound or other soft tissue defect in a subject in need thereof. Such methods generally comprise administering to the subject one or more doses of a therapeutically effective amount of one or more PP compositions. In some embodiments, when multiple doses of the composition(s) are administered, a first dose and a second dose of such multiple doses are administered at least 24 hours to 21 days apart, either by itself or as part of a combination of treatment methods as a protective wound covering. In some embodiments, the wound or soft tissue defect condition is selected from the group consisting of partial and full-thickness wounds, pressure ulcers, venous ulcers, diabetic ulcers, chronic vascular ulcers, tunneled or undermined wounds, surgically created wounds (autograft donor sites), dehisced wounds, trauma wounds (abrasions, lacerations, and skin tears), draining wounds, and partial-thickness burns. In some embodiments of this aspect, one or more doses of the composition(s) is administered by extruding through a syringe tip without needle. In some embodiments, the method results in coverage and protection of the wounded area. In some embodiments, the method results in closure of the open wounded area and filling of the soft tissue defect.

In certain embodiments, rehydration of the dehydrated PP composition to form a self-cohesive mixture generally does not require more than adding a rehydrating solution and simple stirring of the solution/tissue mixture until the mixture has sufficiently rehydrated to be self-cohesive. The rehydrated PP composition is suitable for application directly by hand or tool such as a microspatula, or extrusion from a syringe without a needle.

The inventors have noted that the ease and time of rehydration and subsequent application of dehydrated PP compositions may be affected by a number of factors. Such factors include rehydration technique, rehydration solution, design and composition of the vial, dehydration method, available tools, presence of additives to improve cohesiveness, and degree of rehydration. Any of these factors or combination of these and/or other factors known in the art may be employed to enhance the ease of rehydration of the PP compositions.

In certain embodiments, dehydrated PP compositions may be used without rehydration, such as sprinkled directly from the vial onto a wound in a controlled manner across the wound, pouring the PP compositions first into a second temporary container or tool such as a cup with spout or a spoon, or pouring the PP compositions onto the wound in bulk and then spreading out the PP composition across the wound by hand or with tools or instruments.

The PP compositions described herein can be administered for therapeutic treatments. A “therapeutic amount” or “therapeutically effective amount” administered to an individual suffering from a wound is an amount sufficient to least partially cover the wound. Therapeutic amounts will depend on several factors including size and location of the wound. It is considered well within the skill of the art for one to determine therapeutically effective amounts by routine clinical wound care technique.

The dosages for the PP compositions described herein are from about 35 to 500 mg/vial, for example 35 to about 45 mg, from 75 to 85 mg, from about 155 to 165 mg, from about 315 to about 325 mg, from 300 to 340 mg, from 495 to 505 mg, or from 450 to 550 mg per vial, or any range between, conveniently administered in single or multiple doses depending on the wound site being treated, optionally with follow up doses administered later in time, for example.

The foregoing ranges and timing of doses are merely suggestive, as the number of variables in regard to an individual treatment regime is large. The timing of administration and amount of each dose may vary, depending on several factors including severity and course of the disease or condition, compliance with clinician directions (such as avoiding excessive movement), previous therapy, the patient's health status, age, weight, and response to wound care treatment. It is considered well within the skill of the art for one to determine a therapeutically effective dosing.

The pharmaceutical compositions described herein may be in unit dosage forms suitable for single administration of dosages. In unit dosage form, the formulation is divided into unit doses containing appropriate quantities of the PP composition, e.g., 40 mg, 80 mg, 160 mg, 320 mg, or 500 mg of the PP composition. The unit dosage may be in the form of a package containing discrete quantities of the formulation. A non-limiting example includes the PP composition in vials, trays, or tubes.

In another aspect of the technology, kits containing one or more aliquots of mixed dehydrated APs, CPs, and UCPs with or without additional components and a separate and appropriate amount of solution for rehydrating the mixed placental particles, e.g., 0.9% saline are provided. The kits may include an appropriate device for manipulation and delivery of the PP preparation, e.g., a microspatula, spoon, or cup. The kits may also include instructions for rehydration and administration.

The PP compositions and methods described herein are described in further detail in the following examples. These examples are provided by way of illustration and are not intended to limit the invention in any way.

### EXAMPLES

### Example 1-Production of Dehydrated AP and CP Placental Particles and PP Compositions Comprising a Mixture Thereof

The process described below produced amnion derived particles and chorion derived particles, and a PP composition comprising a mixture thereof and having the following particle size distribution: from about 60% to about 65% of the placental particles being between about 5 um and about 80 um, from about 15% to about 20% of the placental particles being between about 80 um and about 150 um, and from about 15% to about 25% of the placental particles being greater than about 150 um, all percentages being percentages of a total population of placental particles present in the PP composition.
1. The amnion and chorion were separated from each other and the placental disc. (Note: the two components were kept separate throughout the process, then re-combined/mixed in the milling steps 8-9, below.)
2. The membranes were each lightly cleaned of loose blood clots in water with wetted wipes.
3. The amnion and chorion were each, separately, placed into 1 L of 1 M NaCl to decellularize, followed by 2 water rinses. They were then frozen and staged at this step to await process continuation.
4. The two tissues were thawed and then placed, together, into a vessel with a peracetic acid (PAA)-based disinfection solution (about 1 wt % peracetic acid in water, with ethanol and propylene glycol) for 2 hours to disinfect, followed by 8 water rinses. At this stage, all three tissues have been cleaned of remaining blood components that may have elicited an immunogenic response.
5. The tissues were each cut into pieces, with the amnion and chorion folded into approximated 4×4cm pieces. The tissues were then lyophilized separately from one another, to form dry tissue “chips” (the two tissues may alternatively be lyophilized in the same lyophilizer apparatus, just separate from one another).
6. The dry tissue “chips” were removed from the lyophilizer, weighed, and then (optionally) sealed in foil pouches to await process continuation.
7. The amnion and chorion were added together to a mill and milled, as described below, to create the final placental particle mixture. (After completion of Step 9, both placental tissues have been recombined and mixed together.)
   a. The mill was turned on and 5 presses were performed (press fully down then return to top at a steady rate, without waiting at the bottom).
   b. The tissue was stirred, then checked for sufficient milling (completeness) or if tissue needs further milling.
   c. At least once, large unmilled pieces were moved to the top and milled for 2 more presses, then stirred and checked again to determine whether further milling is needed.
   d. Any remaining large unmilled tissue pieces were removed to avoid over-milling.
8. The milled tissue was aliquoted into glass vials from 40 mg-1500 mg, and then fully stoppered and packaged.
   a. Optionally, a second lyophilization step may be performed to further reduce final moisture content with partially stoppered vials, and then fully stoppering the vials and packaging.
   b. Glass vials provide sterility and moisture barrier to preserve over long term shelf-life and clarity to see the tissue; helps clinical user during preparation and retrieval.
   c. The vials may have radiused corners and shoulders (a.k.a. “slanted neck”) to improve the clinical user experience/ability to rehydrate or mix/recover more of the tissue while in the container. (The edge of the bottom of the jar may be flat.) The clinical user will also be able to reach into the container with instrument and recover more of the tissue, as compared to vials having a standard straight neck which often results in tissue getting stuck and inaccessible in the vial. The clinical user may desire to dispense the dry tissue, i.e., without rehydration, and vials having radiused corners and shoulders will facilitate dispensing/recovering more of the dry tissue particles from the vial. The clinical user may want to rehydrate tissue and add saline or other rehydrating solution into the vial.
   d. The vials may be smaller in overall capacity (5 ml vs 10 ml).
   e. The vials can be manufactured from any material.
   f. Optionally, the vials can have screw caps or other type of closure feature.
   g. The vials may serve to preserve sterility and/or moisture barrier.
9. The vials were closed with rubber stoppers and sealed with crimp caps.
   a. The vials may have an alternative closure to a metal crimp cap which the clinical end user needs to tear open. The alternative closure may be a different style metal crimp cap or may be plastic and prevent clinical user from tearing gloves or injury.
10. The vials may be placed in secondary packaging such as foil pouches or trays. The secondary package may or may not serve as a sterility and moisture barrier.
11. The units were then placed into paperboard unit cartons for storage and shipping.
12. Additional packaging features or accessories may be kitted with the tissue unit as an aid for the end user to rehydrate the tissue in the container and retrieve/dispense from the container and apply in a controlled manner to the wound site; minimizing waste/loss of the tissue. For example, a syringe with appropriate distal opening diameter to allow the rehydrated putty-like tissue to dispense.
13. Instructions may be provided to the end user for appropriate rehydration technique such as use of saline, amount, adequate mixing to create desired putty-like consistency.

### Example 2-Production of Dehydrated AP, CP, and UCP Placental Particles and Compositions Comprising Mixtures Thereof

The process described below produced amnion derived particles, chorion derived particles, and umbilical cord particles, and a composition comprising a mixture thereof and having the following particle size distribution: from about 60% to about 65% of the placental particles being between about 5 um and about 80 um, from about 15% to about 20% of the placental particles being between about 80 um and about 150 um, and from about 15% to about 25% of the placental particles being greater than about 150 um, all percentages being percentages of a total population of placental particles present in the PP composition.
1. The amnion, chorion, and umbilical cord were separated from each other and the placental disc. (Note: the three components were kept separate throughout the process, then re-combined/mixed in the milling steps 8-9, below.)
2. The membranes were each lightly cleaned of loose blood clots in water with wetted wipes.
3. The umbilical cord was cut lengthwise (“butterflied”) and scored with a scalpel to expose blood vessels before scraping with a metal elevator to disrupt the vessels and expose the lumen/blood. The cord was then cleaned of loose blood clots in water, (optionally) with wetted wipes. (After completion of Step 3, all three placental tissues are separate.)
4. The amnion, chorion, and umbilical cord were each, separately, placed into 1 L of 1 M NaCl to decellularize, followed by 2 water rinses. They were then frozen and staged at this step to await process continuation.
5. The three tissues were thawed and then placed, together, into a vessel with a peracetic acid (PAA)-based disinfection solution (about 1 wt % peracetic acid in water, with ethanol and propylene glycol) for 2 hours to disinfect, followed by 8 water rinses. At this stage, all three tissues have been cleaned of remaining blood components that may have elicited an immunogenic response.
6. The tissues were each cut into pieces, with the amnion and chorion folded into approximated 4×4cm pieces, and the umbilical cord was cut into 4 cm lengths and further scored as needed to help lay it flat. The tissues were then lyophilized separately from one another, to form dry tissue “chips” (the three tissues may alternatively be lyophilized in the same lyophilizer apparatus, just separate from one another).
7. The dry tissue “chips” were removed from the lyophilizer, weighed, and then (optionally) sealed in foil pouches to await process continuation.
8. The umbilical cord tissue was milled first with a hand-mill to create smaller pieces/particles of umbilical cord.
   a. The mill was turned on and 5 presses were performed (“press”: press mill fully down then return to top at a steady rate, without waiting at the bottom).
   b. The tissue was mixed.
   c. The mill was turned on and 2 presses were performed, followed by 1 3-second hold (“3-second hold”: press mill fully down, hold at bottom for 3 seconds, then return to top).
   d. The tissue was mixed.
   e. The mill was turned on and 2 presses were performed, followed by 1 3-second hold (press fully down, hold at bottom for 3 seconds, then return to top).
   f. The tissue was mixed.
9. The amnion and chorion were added to the cord and further milled, as described below, to create the final placental particle mixture. (After completion of Step 9, all three placental tissues have been recombined and mixed together.)
   a. The mill was turned on and 5 presses were performed (press fully down then return to top at a steady rate, without waiting at the bottom).
   b. The tissue was stirred, then checked for sufficient milling (completeness) or if tissue needs further milling.
   c. At least once, large unmilled pieces were moved to the top and milled for 2 more presses, then stirred and checked again to determine whether further milling is needed.
   d. Any remaining large unmilled tissue pieces were removed to avoid over-milling.
10. The milled tissue was aliquoted into glass vials from 40 mg-1500 mg, and then fully stoppered and packaged.
   a. Optionally, a second lyophilization step may be performed to further reduce final moisture content with partially stoppered vials, and then fully stoppering the vials and packaging.
   b. The glass vials are as described in Example 1 above (Step 8, parts b-g) and provide sterility and moisture barrier to preserve over long term shelf-life and clarity to see the tissue; helps clinical user during preparation and retrieval.
11. The vials were closed with rubber stoppers and sealed with crimp caps.
   a. The vials may have an alternative closure to a metal crimp cap which the clinical end user needs to tear open. The alternative closure may be a different style metal crimp cap or may be plastic and prevent clinical user from tearing gloves or injury.
12. The vials may be placed in secondary packaging such as foil pouches or trays. The secondary package may or may not serve as a sterility and moisture barrier.
13. The units were then placed into paperboard unit cartons for storage and shipping.
14. Additional packaging features or accessories may be kitted with the tissue unit as an aid for the end user to rehydrate the tissue in the container and retrieve/dispense from the container and apply in a controlled manner to the wound site; minimizing waste/loss of the tissue. For example, a syringe with appropriate distal opening diameter to allow the rehydrated putty-like tissue to dispense.
15. Instructions may be provided to the end user for appropriate rehydration technique such as use of saline, amount, adequate mixing to create desired putty-like consistency.

### Example 3-Placental Particle Composition Preparation (APs, CPs, and UCPs)

The PP composition is derived from AM, CM, and UC components of placental tissue recovered from a single donor. Prior to processing, donated placental tissue is aseptically recovered from a live full-term birth.

Donor placental tissue processing is initiated within 120 hours from the time of recovery. The placental tissue is then separated (i.e., amnion, chorion, and umbilical cord, which is measured at time of acquisition and later dissected to expose blood vessels), cleaned to remove blood clots, rinsed with phosphate buffered saline (3-5 minutes per rinse at room temperature).

The separated placental tissues (i.e., amnion, chorion, and umbilical cord) are each cut into smaller pieces or strips, e.g., 4 cm pieces or strips (the umbilical cord being further scored as needed to help lay it flat), and separately lyophilized to produce dehydrated “chips” of amnion and chorion, and dehydrated strips of the thicker, denser umbilical cord (UC chips).

The lyophilized UC strips are separately milled first (i.e., “pre-milled”), such as with a hand-mill or freezer mill, to intermediate pieces of umbilical cord, which will ultimately yield particles of similar size to the milled amnion and chorion chips.

### Example 3A-Pre-Mixed Dehydrated PP Composition

At this point, the lyophilized amnion, chorion, and umbilical cord chips may each be weighed to obtain desired predetermined quantities for combination into a mixture of desired proportions of amnion, chorion, and umbilical cord. Whether weighed or not, the lyophilized amnion and chorion chips are added to the lyophilized pre-milled umbilical cord pieces and the combined tissues are then further milled to create a PP composition, i.e., a mixture of dehydrated amnion, chorion, and umbilical cord particles (i.e., APs, CPs, and UCPs, respectively).

In some embodiments, the dry weight percentages of each of the APs, CPs, and UCPs in the PP composition are expected to be, for example, without limitation: about 10-30 wt % APs, about 35-75 wt % CPs, and about 15-45 wt % UCPs. This PP composition is then placed into single-use glass vials (aliquoted by weight) that are lyophilized a final time, stoppered and sealed. Each vial of the resulting PP composition is then packaged in a foil pouch and sealed prior to terminal sterilization, for example, by electron beam irradiation.

Alternatively, lyophilized amnion, chorion, and pre-milled umbilical cord pieces produced as set forth above, are each milled separately, using a hand-mill or other milling apparatus such as a freezer mill to produce separate APs, CPs, and UCPs, which are then measured (weighed) to provide desired quantities of the dehydrated PPs. In some embodiments, the measured separate quantities of APs, CPs, and UCPs are combined to produce a PP composition, which may then be aliquoted by weight into individual vials and, optionally, lyophilized again and packaged as described above.

### Example 3B-Kit Containing Separate Dehydrated APs, CP, and UCPs, Combined at Time of Use

In other embodiments, separate measured quantities of APs, CPs, UCPs and, optionally, CPPs, are each placed into at least one vial or other container. In other words, each of the vials or other containers may include more than one of the measured quantities of APs, CPs, UCPs and, optionally, CPPs. Furthermore, the separate containers may be subjected to one or more of lyophilizing, packaging, and terminal sterilization, and then collected and placed into a carton, tray, or other container together, to provide a kit which includes at least one container with one or more of the four types of dehydrated PPs (APs, CPs, UCPs, CPPs) for combination at or within a reasonable time prior to the time of use. At the point of care, a health practitioner or other user would open the kit, open each of the separate containers and combine the APs, CPs, UCPs and, optional CPPs, to form a PP composition, with or without a hydrating fluid, carrier or other materials.

### Example 4-Production of Dehydrated Placental Particles and Compositions Comprising Mixtures Thereof Without Pre-Milling

The process described below produces amnion derived particles, chorion derived particles, and umbilical cord particles, and a composition comprising a mixture thereof and having following the particle size distribution: from about 60% to about 65% of the placental particles being between about 5 um and about 80 um, from about 15% to about 20% of the placental particles being between about 80 um and about 150 um, and from about 15% to about 25% of the placental particles being greater than about 150 um, all percentages being percentages of a total population of placental particles present in the PP composition.
1. The amnion, chorion, and umbilical cord are separated from each other and the placental disc. (Note: the three components are kept separate throughout the process, then re-combined/mixed after the milling steps 8-9, below.)
2. The membranes are each lightly cleaned of loose blood clots in water with wetted wipes.
3. The umbilical cord is cut lengthwise (“butterflied”) and scored with a scalpel to expose blood vessels before scraping with a metal elevator to disrupt the vessels and expose the lumen/blood. The cord is then cleaned of loose blood clots in water, (optionally) with wetted wipes. (After completion of Step 3, all three placental tissues are separated.)
4. The amnion, chorion, and umbilical cord are each, separately, placed into 1 L of 1 M NaCl to decellularize, followed by 2 water rinses. They are then frozen and staged at this step to await process continuation.
5. The three tissues are thawed and then placed, together, into a vessel with a peracetic acid (PAA)-based disinfection solution to disinfect, followed by 8 water rinses. At this stage, all three tissues have been cleaned of remaining blood components that may have elicited an immunogenic response.
6. The tissues are each cut into pieces, with the amnion and chorion folded into approximated 4×4cm pieces, and the umbilical cord cut into 4 cm lengths and further scored as needed to help lay it flat. The tissues are then lyophilized separately from one another, to form dry tissue “chips” (the three tissues may be lyophilized in the same lyophilizer apparatus, just separate from one another).
7. The dry tissue “chips” are removed from the lyophilizer, weighed, and then (optionally) sealed in foil pouches to await process continuation.
8. The umbilical cord tissue is milled with a hand-mill to create particles of umbilical cord of the desired size range.
9. The amnion and chorion are milled with a hand-mill to create particles of amnion and chorion of the desired size range.
10. The umbilical cord particles are combined with the amnion and chorion particles to create the final placental particle mixture. (After completion of Step 10, all three placental tissues have been recombined and mixed together.)
11. The milled tissue is aliquoted into glass vials from 40 mg-1500 mg, partially stoppered, and then lyophilized again to reduce final residual moisture before fully stoppering under vacuum.
   a. The second lyophilization step may be removed and the process will proceed with fully stoppering the vials and packaging.
   b. The glass vials are as described in Example 1 above (Step 8, parts b-g) and provide sterility and moisture barrier to preserve over long term shelf-life and clarity to see the tissue; helps clinical user during preparation and retrieval.
12. The vials are closed with rubber stoppers and sealed with crimp caps.
   a. The vials may have an alternative closure to a metal crimp cap which the clinical end user needs to tear open. The alternative closure may be a different style metal crimp cap or may be plastic and prevent clinical user from tearing gloves or injury.

### Example 5-Production of Dehydrated AP, CP, UCP, and CPP Placental Particles and Compositions Comprising Mixtures Thereof

The process described below produced amnion derived particles, chorion derived particles, umbilical cord particles, and chorionic plate particles, and a composition comprising a mixture thereof and having the following particle size distribution: from about 60% to about 65% of the placental particles being between about 5 um and about 80 um, from about 15% to about 20% of the placental particles being between about 80 um and about 150 um, and from about 15% to about 25% of the placental particles being greater than about 150 um, all percentages being percentages of a total population of placental particles present in the PP composition.

### Separation, Cleaning, Decellularization, Disinfection, and Lyophilization of Amnion, Chorion, Chorionic Plate, and Umbilical Cord:

1. The amnion, chorion, and umbilical cord were separated from each other and the placental disc.
2. Using a scalpel, elevator, or other scraping tool, the edges of the placental disc tissue were scraped away from the maternal facing surface of the chorionic plate.
3. The placental disc tissue was further scraped away from the chorionic plate, while scissors or other cutting instruments were used to cut the chorionic villi extending from the chorionic plate into the placental disc tissue. After fully separating the chorionic plate from the placental disc, the chorionic plate was placed into water. (After completion of Step 3, all four desired placental tissue components were separate.)
4. The amnion and chorion membranes were each lightly cleaned of loose blood clots in water with wetted wipes.
5. The umbilical cord was cut lengthwise (“butterflied”) and scored with a scalpel to expose blood vessels before scraping with a metal elevator to disrupt the vessels and expose the lumen/blood. The cord was then cleaned of loose blood clots in water, (optionally) with wetted wipes.
6. The chorionic plate was trimmed of protruding villi and scraped to remove residual placental disc tissue. Any visible vessels were scored with a scalpel, as necessary to expose the blood vessels before scraping with a metal elevator to disrupt the vessels and expose the lumen/blood, and then the chorionic plate was cleaned in water with wetted wipes as needed.
7. The amnion, chorion, umbilical cord, and chorionic plate were each placed into a flask with 1 L of 1 M NaCl solution.
8. The flasks were placed on an orbital shaker at room temperature and agitated for 1.75 hours.
9. The NaCl solution was replaced with water and the tissue agitated in flasks for 5 minutes.
10. The water was replaced with fresh water and the tissue was agitated in flasks for 5 minutes.
11. The tissues were each drained of excess water and packaged then frozen for storage.
12. The amnion, chorion, chorionic plate, and umbilical cord were thawed and then placed together in a vessel for a chemical disinfection process: disinfection solution (about 1 wt % peracetic acid in water, with ethanol and propylene glycol) for 2 hours, then 8 water rinses (4× 5 min, 2× 10 min, 2× 15 min)
13. The tissues were cut into pieces and lyophilized.

### Milling of Amnion, Chorion, Chorionic Plate, and Umbilical Cord to Produce Particles:

1. The chorionic plate and umbilical cord tissue pieces were added into the milling cup for a pre-milling sequence.
   a. The mill was turned on and 5 presses were performed (“press”: press mill fully down then return to top at a steady rate, without waiting at the bottom).
   b. The tissue was mixed.
   c. The mill was turned on and 2 presses were performed, followed by 1 3-second hold (“3-second hold”: press mill fully down, hold at bottom for 3 seconds, then return to top).
   d. The tissue was mixed.
   e. The mill was turned on and 2 presses were performed, followed by 1 3-second hold (press fully down, hold at bottom for 3 seconds, then return to top).
   f. The tissue was mixed.
2. The amnion and chorion were added to the pre-milled chorionic plate and umbilical cord tissue pieces.
3. The mill was turned on and 5 presses were performed (press fully down then return to top at a steady rate, without waiting at the bottom).
4. The tissue was stirred, then checked for sufficient milling (completeness) or if tissue needs further milling.
5. At least once, large unmilled pieces were moved to the top and milled for 2 more presses, then stirred and checked again to determine whether further milling is needed.
6. Any remaining large unmilled tissue pieces were removed to avoid over-milling.
7. The milled tissue was aliquoted into containers by weight (alternatively, may be aliquoted by volume).

### Example 6-Particle Size Analysis via Imaging and Software

Particle size distribution was analyzed for each of the following 3 embodiments of the PP Compositions:
“2-Component PP Composition”—comprising a mixture of APs and CPs in accordance with the present disclosure and prepared according to Example 1 above; and
“3-Component PP Composition”—comprising a mixture of APs, CPs, and UCPs in accordance with the present disclosure and prepared according to Example 2 above; and
“4-Component PP Composition”—comprising a mixture of dehydrated APs, CPs, UCPs, and CPPs in accordance with the present disclosure and prepared according to Example 5 above.
1. For 3 different donors (batches) of each of the 2-, 3-, and 4-Component PP Compositions (as listed above for this experiment), a sample of each was evenly sprinkled onto each of 5 glass slides and then covered lightly with clear tape.
   a. For the 2-Component PP Composition, 2 slides were prepared from 1 donor and 1 slide was prepared from a second donor.
2. Each slide was placed on a microscope with a 2× objective, with ambient lights on and the microscope light off such that the particles appeared bright on a dark background.
3. The slides were imaged, moving from viewing field to field to avoid overlapping and recounting the same particle.
4. For the 3 different donors of Mini Membrane w/umbilical cord and for Mini Membrane w/umbilical cord & chorionic plate, multiple microscope fields were imaged across the 5 slides per donor for semi-automated particle size analysis using open source ImageJ/Fiji software (https://imagej.net/software/fiji/).
   a. For Mini Membrane w/o Umbilical Cord (original), fields were imaged for each slide and reported as per slide (2 slides from 1 donor, 1 slide from a second donor) vs. aggregated across slides per donor.
5. Each image was opened in Fiji, converted to 8-bit and then the brightness threshold adjusted such that the particles were visible against the background. A global scale was set.
6. The Analyze Particle function was then used to identify and measure each particle, providing each particle count's area and feret diameter (maximum diameter across a particle), also known as caliper diameter.
7. The feret diameter was used as the parameter for determining particle size, using Microsoft Excel to generate a histogram graphing their frequency as % of total count for each size category.

The results of the foregoing particle size analysis and distribution for each of the each of the 2-, 3-, and 4-Component PP Compositions are shown in FIGS.

### Example 7. Enzyme Degradation Assay—Compare PTP Prior Art, 3-Component PP Composition (AM, CM, UC), and 4-Component PP Composition (AM, CM, UC, CP)

This experiment compared the enzymatic degradation rates of the following three placental particulate compositions by exposing each to physiologically comparable enzymatic solutions and measuring the mass loss for each, which was proportional to the enzymatic degradation for each composition.

“PTP Composition”—a prior art placental particulate composition comprising particles derived from amnion, chorion, and umbilical cord and prepared according to the process disclosed in Example 1 of U.S. Patent Application Publication No. 2021/0154240, as reproduced in relevant part below;

### PTP Composition Preparation

Donor placental tissue processing was initiated within 72 hours from the time of recovery. Prior to processing, tissue was inspected and an incoming bioburden sample was collected. The donated placental tissue was then separated (i.e., amnion, chorion, and umbilical cord, which was measured at time of acquisition and later dissected to remove blood vessels), cleaned to remove blood clots, rinsed with phosphate buffered saline (3-5 minutes per rinse at room temperature), cut into smaller pieces, e.g., 4 cm strips and lyophilized. Following lyophilization, the tissue was cut again to facilitate cryogenic milling. The individual milled placental tissues were weighed, combined, and sieved between 20 and 150 um sieves to produce placental tissue particulate. The tissues were combined in ratios that maximized the yield of the PTP composition. The dry weight percentages of each placental tissue in the combined PTP are shown in Table 1. This placental tissue particulate was then filled into single-use Type 1 glass vials (aliquoted by weight) that were lyophilized a final time, stoppered and sealed. Each vial of PTP was then packaged in a foil pouch and sealed prior to terminal sterilization by electron beam irradiation.

“2-Component PP Composition”—comprising a mixture of APs and CPs in accordance with the present disclosure and prepared according to Example 1 above; and

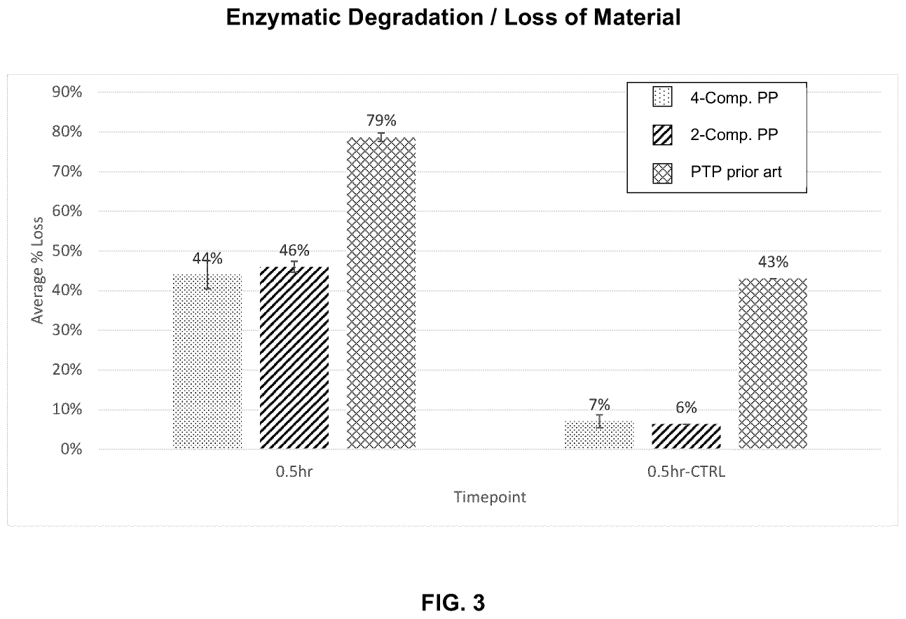

“4-Component PP Composition”—comprising a mixture of dehydrated APs, CPs, UCPs, and CPPs in accordance with the present disclosure and prepared according to Example 5 above.
1. For each sample of the above-listed compositions:
   a. An empty 2 mL tube was weighed and the weight was recorded.
   b. Approximately 40 mg tissue was added to the tube and the exact mass was recorded.
2. One sample of each product type was tested as a non-enzyme control.
3. 0.1 M Tris-HCl with CaCl2 stock solution was prepared as follows:
   a. Dilute 1 M Tris-HCl to 0.1 M: Add 4 mL of 1 M Tris to 36 mL of water
   b. Add CaCl2 to 3 mM: 0.0144 g CaCl2 added to 1 M Tris-HCl
   c. A portion of 0.1 M Tris-HCl with CaCl2 stock solution was set aside for non-enzyme controls (ie. 5 mL)
4. 50 U/mL Collagenase II was prepared as follows:
   a. Collagenase II powder was added to remaining 0.1 M Tris-HCl with CaCl2 solution based on activity on bottle (275 U): 0.0066 g of Collagenase II powder added to 35 mL of stock solution.
5. 1 mL collagenase II solution was added to each test sample tube and 1 mL of stock solution was added to each non-enzyme control tube. Sample tubes were vortexed to mix after solution was added.
6. Sample tubes were incubated at 37° C. on an orbital shaker at 150 rpm for 30 minutes.
7. Samples were removed from incubation and centrifuged for 5 min. at 10,000 rpm.
8. Supernatant liquid was aspirated off taking care not to disturb or remove any tissue.
9. A series of three rinses were performed comprised of the following steps:
   a. 1 mL of water was added to each sample
   b. Each sample was vortexed to mix
   c. The samples were centrifuge for 5 minutes at 10,000 rpm.
   d. Supernatant liquid was aspirated off taking care not to disturb or remove any tissue.
10. The lid of each sample was closed and a hole was punctured through it using a 16 g needle.
11. The samples were freeze dried to remove all residual moisture.
12. Following completion of drying the samples were each weighed and the weights were recorded as the post-drying total mass.
13. Post-drying tissue mass was calculated for each sample by subtracting original empty tube mass from the post-drying total mass.
14. The percent of tissue lost post-degradation was calculated by dividing the post-drying tissue mass by the starting tissue mass for each sample, subtracting from 1, and multiplying by 100.
15. Results are shown in FIG. 3—There was more degradation and material loss for the PTP Composition (prior art) than for either of the 2-Component and 4-Component PP Compositions. Further, both of the 2-Component and 4-Component PP Compositions showed similar levels of degradation and mass loss.

### Example 8—Implant Washout Simulation—Compare PTP Prior Art, and 4-Component PP Composition (AM, CM, UC, CP)

The goal of this experiment was to determine the resistance of each formulation to being washed out by fluid when applied (such as after administration to a treatment site). Each composition tested was incubated in fluid for full hydration and then multiple washes were employed and the remaining product was determined.
“PTP Composition”—a prior art placental particulate composition comprising particles derived from amnion, chorion, and umbilical cord and prepared according to the process disclosed in Example 1 of U.S. Patent Application Publication No. 2021/0154240, as previously reproduced in relevant part in Example 7 above;
“4-Component PP Composition”—comprising a mixture of dehydrated APs, CPs, UCPs, and CPPs in accordance with the present disclosure and prepared according to Example 4 above.
1. For each sample prepared for testing:
   a. An empty 2 mL tube was weighed and the weight was recorded.
   b. Approximately 40 mg tissue was added to the tube and the exact mass was recorded.
2. 1 mL of non-enzymatic solution was added to tube. Sample tubes were vortexed to mix after solution was added.
3. Sample tubes were incubated at 37° C. on an orbital shaker at 150 rpm for 30 minutes.
4. Samples were removed from incubation and centrifuged for 5 min. at 10,000 rpm.
5. Supernatant liquid was aspirated off taking care not to disturb or remove any tissue.
6. A series of three water washes were performed comprised of the following steps:
   a. 1 mL of water was added to each sample
   b. Each sample was vortexed to mix
   c. The samples were centrifuge for 5 minutes at 10,000 rpm.
   d. Supernatant liquid was aspirated off taking care not to disturb or remove any tissue.
7. The lid of each sample was closed and a hole was punctured through it using a 16 g needle.
8. The samples were freeze dried to remove all residual moisture.
9. Following completion of drying the samples were each weighed and the weights were recorded as the post-drying total mass.
10. Post-drying tissue mass was calculated for each sample by subtracting original empty tube mass from the post-drying total mass.
11. The percent of tissue lost was calculated by dividing the post-drying tissue mass by the starting tissue mass for each sample, subtracting from 1, and multiplying by 100.

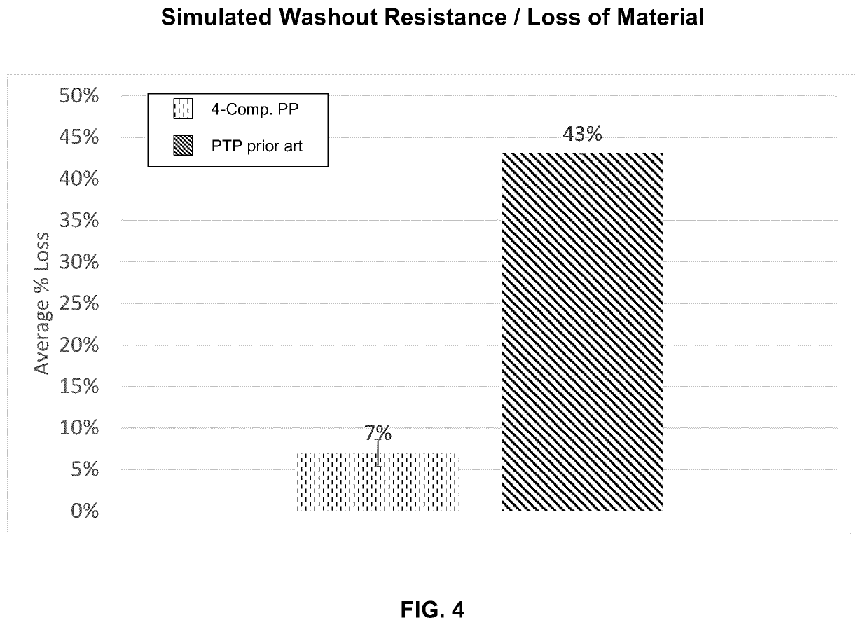

The results are shown in FIG. 4.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

1. A composition comprising placenta-derived particles (PP composition) which comprises a mixture of one or more of: amnion membrane derived particles (APs), chorionic membrane derived particles (CPs), umbilical cord derived particles (UCPs), and chorionic plate derived particles (CPPs).

2. The composition of claim 1, wherein the PP composition includes from about 10% to about 30% of the total population placental particles being greater than about 150 um.

3. The composition of claim 2, wherein the PP composition has the following particle size distribution:
from about 50% to about 80% of the placental particles being between about 5 um and about 80 um, and
from about 10% to about 30% of the placental particles being from about 80 um to about 150 um,
wherein the aforesaid percentages are percentages of a total population of placental particles present in the PP composition.

4. The composition of claim 3, wherein the PP composition has the following particle size distribution:
from about 50% to about 70% of the placental particles being between about 5 um and about 80 um,
from about 15% to about 25% of the placental particles being between about 80 um and about 150 um, and
from about 15% to about 25% of the placental particles being greater than about 150 um,
wherein the aforesaid percentages are percentages of a total population of placental particles present in the PP composition.

5. The composition of claim 1, wherein the dry weight percentages of each of the APs, CPs, UCPs, and CPPs present in the PP composition are:
from about 5 wt % to about 95 wt % APs,
from about 5 wt % to about 95 wt % CPs,
from about 0 wt % to about 90 wt % UCPs, and
from about 0 wt % to about 90 wt % CPPs,
based on the total weight of the PP composition.

6. The composition of claim 5, wherein the dry weight percentages of each of the APs, CPs, UCPs, and CPPs present in the PP composition are:
from about 10 wt % to about 30 wt % of APs,
from about 30 wt % to about 75 wt % of CPs,
from about 5 wt % to about 50 wt % UCPs, and
from about 5 wt % to about 50 wt % CCPs,
based on the total weight of the PP composition.

7. The composition of claim 5, wherein the PP composition comprises at least APs, CPs, and either UCPs or CPPs, and the dry weight percentages of each of the APs, CPs, and either UCPs or CPPs present in the PP composition are: about 10-30% by weight (wt %) of APs, about 35-75 wt % of CPs, and about 15-45 wt % of UCPs or CPPs, based on the total weight of the PP composition.

8. The composition of claim 5, wherein the PP composition comprises APs, CPs, UCPs, and CPPs, and the dry weight percentages of each of the APs, CPs, UCPs, and CPPs in the PP composition are: about 10-30 wt % APs, about 10-50 wt % CPs, about 10-50 wt % UCPs and about 10-50 wt % CPPs, based on the total weight of the PP composition.

9. A method for producing placental particles and mixtures thereof, from a placenta which includes at least amnion, chorion, and umbilical cord, generally comprises the steps of:
(A) separating one or more of the amnion, the chorion, and the umbilical cord from each other;
(B) cleaning each of the separated amnion and chorion, separately and lightly to remove loose blood clots from each placental tissue without causing damage to the tissues;
(C) cutting and cleaning the umbilical cord to facilitate contacting the umbilical cord with processing agents and removing additional blood clots and blood from the umbilical cord;
(D) decellularizing one or more of the amnion, chorion, and umbilical cord, separately from each other;
(E) optionally, freezing one or more of the decellularized amnion, chorion, and umbilical cord, and thawing each when processing is re-commenced;
(F) optionally, disinfecting one or more of the amnion, chorion, and umbilical cord, separately or together;
(G) cutting each of the one or more amnion, chorion, and umbilical cord into pieces having sizes suitable for milling in a milling apparatus,
(H) dehydrating each of the one or more of amnion, chorion, and umbilical cord, separately from each other, to produce dry amnion (AM) chips, dry chorion (CM) chips, and dry umbilical (UC) cord chips;
(I) optionally, storing each of the dry AM chips, the dry CM chips, and the dry UC chips, separately from each other, in sealed foil pouches until processing is continued;
(J) pre-milling the dry UC chips to form intermediate sized pre-milled UC chips which are smaller than the dry UC chips forming during the dehydrating step (H)
(K) combining together desired quantities of each of the dry AM chips, the dry CM chips, and the pre-milled UC chips, to produce a placental tissue mixture comprising predetermined proportions of each of the dry AM, CM, and UC chips in the mixture; and
(L) milling the placental tissue mixture of dry AM, CM, and UC chips and pieces to produce a placental particle composition comprising a mixture of dehydrated amnion derived particles (APs), chorion derived particles (CPs), and umbilical cord derived particles (UCPs), wherein the placental particle composition comprises up to about 30% of placental particles having particle size greater than 150 um.

10. The method of claim 9, further comprising sterilizing the placental particle composition by applying one or more techniques selected from: by exposing the composition to gamma radiation, e-beam radiation, UV light, ethylene oxide, and combinations thereof.

11. The method of claim 9, wherein the intermediate size of the pre-milled dry UC chips produced by the pre-milling step (J) is smaller than the dry UC chips produced during dehydrating step (H).

12. The method of claim 9, wherein the placenta further includes a placental disk having the chorionic plate lying thereon, and wherein:
the step of (A) separating the chorionic plate includes scraping edges of the placental disk on a maternal facing surface of the chorionic plate, scraping a side of the placental disk opposite the chorionic plate to further expose the chorionic plate and chorionic villi extending therefrom, and cutting away the chorionic villi, and placing the chorionic plate into water;
the step of (B) cleaning includes cleaning the separated chorionic plate, separately and lightly to remove loose blood clots without causing damage to the tissues;
the step of (D) decellularizing includes decellularizing the chorionic plate;
the step of (G) cutting includes cutting the chorionic plate into pieces having sizes suitable for milling in a milling apparatus,
the step of (H) dehydrating includes dehydrating the chorionic plate to produce dry chorionic plate (CP) chips;
the step of (K) combining includes also combining a desired quantity of the dry CP chips to produce a mixture comprising predetermined proportions of each of the dry AM, CM, UC, and CP chips and pieces, respectively, in the mixture; and
the step of (L) milling the mixture produces a placental particle composition comprising a mixture of dehydrated APs, CPs, UCPs and chorionic plate particles (CPPs).

13. The method of claim 12, wherein:
the step of (J) pre-milling comprises combining the dry UC chips and the dry CP chips to form mixture of dry UC chips and dry CP chips, and pre-milling the mixture to produce a mixture of intermediate sized pre-milled UC chips and intermediate sized pre-milled CP chips, which are smaller than the dry UC and CP chips formed during the dehydrating step (H); and
the step of (K) combining includes combining desired quantities of each of the dry AM chips and the dry CM chips to the mixture of pre-milled UC and CP chips, to produce a mixture comprising predetermined proportions of each of the dry AM, CM, UC, and CP chips in the mixture.

14. The method of claim 13, wherein the step of (J) pre-milling comprises:
performing 5 presses using a press mill, wherein each press comprises a steady rate of return of the press mill from its fully down position to its top position, without a pause at the fully down position, then mixing the UC and CP tissue;
performing 2 presses are performed, wherein each press includes a 3-second hold while the mill is at the fully down position, then mixing the UC and CP tissue; and
performing 2 presses are performed, wherein each press includes a 3-second hold while the mill is at the fully down position, then mixing the UC and CP tissue.

15. The method of claim 13, wherein the step of (L) milling the placental tissue mixture, comprising predetermined proportions of each of the dry AM, CM, UC, and CP chips, comprises:
performing 5 presses using a press mill, wherein each press comprises a steady rate of return of the press mill from its fully down position to its top position, without a pause at the fully down position, then mixing the UC and CP tissue,
determining whether further milling is necessary, and conducting further milling if it is determined to be necessary; and
optionally, moving large unmilled tissue pieces to a top section of the placental tissue mixture being milled, then continuing milling of the mixture, or removing the large unmilled tissue pieces from the placental tissue mixture to avoid over milling.
